(19) United States
(12) Patent Application Publication
(10) Pub. No.: US 2024/0212789 A1
(43) Pub. Date: Jun. 27, 2024

(54) METHOD AND SYSTEM FOR PREDICTING A PHENOTYPIC FEATURE OF A HOST BASED ON A MICROBIOME OF THE HOST

(71) Applicant: NEC Laboratories Europe GmbH, Heidelberg (DE)

(72) Inventors: Filippo GRAZIOLI, Heidelberg (DE); Andrea MEISER, Heidelberg (DE); Giampaolo PILEGGI, Heidelberg (DE); Raman SIARHEYEU, Heidelberg (DE)

(21) Appl. No.: 18/288,833

(22) PCT Filed: Aug. 3, 2021
(86) PCT No.: PCT/EP2021/071705

§ 371 Date: Oct. 30, 2023

(30) Foreign Application Priority Data
May 19, 2021 (EP) 21174837.1

(51) Int. Cl. G16B 20/00; G16B 40/20

(52) U.S. Cl. CPC G16B 20/00; G16B 40/20

(57) ABSTRACT

A method for predicting a phenotypic feature of a host based on a microbiome of the host by means of a data processing system includes providing or collecting microbiome-specific data and host-specific data, joining the microbiome-specific data and the host-specific data by computing a joint representation, and predicting the phenotypic feature on the basis of the joint representation by means of at least one machine learning model or machine learning algorithm. The method can be used to support the development of optimized immunotherapies.

This application is a U.S. National Phase application under 35 U.S.C. § 371 of International Application No. PCT/EP2021/071705, filed on Aug. 3, 2021, and claims benefit to European Patent Application No. EP 21174837.1, filed on May 19, 2021. The International Application was published in English on Nov. 24, 2022 as WO 2022/242886 A1 under PCT Article 21(2).

### FIELD

The present invention relates to a method for predicting a phenotypic feature of a host based on a microbiome of the host by means of a data processing system.

Further, the present invention relates to a data processing system for carrying out the above method.

### BACKGROUND

Corresponding prior art documents are listed as follows:
[1] Cho, I. & Blaser, M. J. The human microbiome: at the interface of health and disease. Nature Reviews Genetics 13, 260 (2012).
[2] Huttenhower, C. et al. Structure, function and diversity of the healthy human microbiome. Nature 486, 207 (2012).
[3] McQuade, J. L., Daniel, C. R., Helmink, B. A. & Wargo, J. A. Modulating the microbiome to improve therapeutic response in cancer. The Lancet Oncology 20, e77-e91 (2019).
[4] Eloe-Fadrosh, E. A. & Rasko, D. A. The human microbiome: from symbiosis to pathogenesis. Annual Review of Medicine 64, 145-163 (2013).
[5] Oh, M. & Zhang, L. DeepMicro: deep representation learning for disease prediction based on microbiome data. Nature Scientific Reports 10, 6026 (2020).
[6] Alemi, A. A., Fischer, I., Dillon, J.V. & Murphy, K. Deep variational information bottleneck. arXiv preprint arXiv:1612.00410 (2016).
[7] Tishby, N., Pereira, F.C. & Biale, W. The information bottleneck method. Proc. of the 37th Annual Allerton Conf. on Communication, Control and Computing, pp. 368-377 (1999).
[8] Wu, M. & Goodman, N. Multimodal generative models for scalable weakly-supervised learning. arXiv preprint arXiv: 1802.05335 (2018)
[9] LaPierre, N., Ju, C. J-T., Zhou, G. & Wang, W. Metapheno: A critical evaluation of deep learning and machine learning in metagenome-based disease prediction. Methods, 166:74-82 (2019)
[10] Aryal, S., Alimadadi, A., Manandhar, I., Joe, B. & Cheng, X. Machine learning strategy for gut microbiome-based diagnostic screening of cardiovascular disease. Hypertension, 76(5): 1555-1562 (2020).
[11] Reiman, D., Metwally, A. A., Sun, J. & Dai, Y. PopPhy-CNN: a phylogenetic tree embedded architecture for convolutional neural networks to predict host phenotype from metagenomic data. IEEE Journal of Biomedical and Health Informatics, 24(10):2993-3001(2020).

The human microbiota consists of a wide variety of microorganisms which live in and on our body. Such communities of microorganisms, composed by trillions of microbes of highly diverse species, constitute highly complex and diverse ecosystems which interact with the host system, i.e. the human body. Recent studies have shown that the human microbiota plays a key role in human health and diseases, see [1]. Humans benefit from the presence of these microorganisms, as they enable important chemical processes. For example, the microbiota plays a crucial role in maintaining homeostasis, developing the host immune system, protecting the integrity of mucosal barriers and harvesting nutrients which would otherwise be inaccessible, see [1], [2]. There is also empirical evidence of how altered states of the microbiota can contribute to carcinogenesis and consistently affect therapeutic response, see [3]. These emerging discoveries lead to the conclusion that any system that attempts to predict host's metabolism- and immunity-related phenotypic features would be incomplete if the microbiota is not addressed. To further substantiate this, [4] shows the potential use of the microbiota as a predictor for various diseases.

The human body is populated by a large abundance of microorganisms, living with us on our skin, mucosal membranes and inside us, particularly in the large intestine. Humans and their microbes co-evolved, and adapted living together. The human body consists of approx. 30 trillion human cells plus 39 trillion microbial cells. On a genetic level, the human genes comprise of approx. 20,000 genes, whilst our microbiota contains between approx. 2-20 million genes. The influence of these microbes on our health, their communication with our human cells, in particular their influence on our immune system, has long been unknown.

High-throughput sequencing technologies has allowed scientists to capture a highly descriptive snapshot of microbial communities. 16S rRNA gene sequencing technology allows for a cost-effective profiling of the most common components of the human microbiome. Shotgun metagenomic sequencing technology can provide a higher resolution profile at the strain level. As the cost of shotgun metagenomic sequencing keeps decreasing and the resolution increasing, high-quality microbiome data is becoming increasingly more accessible, and this will inevitably lead to higher diffusion of microbiome-based applications in the human healthcare sector.

### SUMMARY

In an embodiment, the present disclosure provides a method for predicting a phenotypic feature of a host based on a microbiome of the host by a data processing system. The method includes providing or collecting microbiome-specific data and host-specific data, joining the microbiome-specific data and the host-specific data by computing a joint representation, and predicting the phenotypic feature on the basis of the joint representation using at least one machine learning model or machine learning algorithm. The method can be used to support the development of optimized immunotherapies.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 shows in a diagram a depiction of a system according to an embodiment of the invention;

FIG. 2 shows in a diagram a fusion of single-modality latent distributions to compute a joint posterior according to an embodiment of the invention;

FIG. 3 shows in a diagram a depiction of building blocks of an embodiment of the invention; and

FIG. 4 shows in a diagram a depiction of a system in its “Health Trajectory Prediction” implementation according to an embodiment of the invention.

### DETAILED DESCRIPTION

In accordance with an embodiment, the present invention improves and further develops a method for predicting a phenotypic feature of a host and a corresponding system for providing an extra efficient and accurate prediction of a phenotypic feature of a host by simple means.

In accordance with an embodiment, the present invention provides a method for predicting a phenotypic feature of a host based on a microbiome of the host by means of a data processing system, comprising the following steps:
providing or collecting microbiome-specific data and host-specific data;
joining said microbiome-specific data and said host-specific data by computing a joint representation; and
predicting the phenotypic feature on the basis of the joint representation by means of at least one machine learning model or machine learning algorithm.
   Further, in accordance with an embodiment, the present invention provides a data processing system for carrying out a method for predicting a phenotypic feature of a host based on a microbiome of the host, comprising:
providing or collecting means for providing or collecting microbiome-specific data and host-specific data;
joining means for joining said microbiome-specific data and said host-specific data by computing a joint representation; and
predicting means for predicting the phenotypic feature on the basis of the joint representation by means of at least one machine learning model or machine learning algorithm.

According to the invention it has been recognized that it is possible to solve the aforementioned by joining or combining microbiome-specific and host-specific data in a suitable way, wherein the host can be a human body or patient. It has been further recognized that such a suitable way is provided by joining said microbiome-specific data and said host-specific data by computing a joint representation with a suitable data processing system. On the basis of this joint representation the phenotypic feature can be predicted by means of at least one machine learning model or machine learning algorithm. Corresponding research has shown that predicting results can be provided by the method and system according to the invention very efficiently and with high accuracy.

Thus, on the basis of the invention a method for predicting a phenotypic feature of a host and a corresponding data processing system with an extra efficient and accurate prediction of a phenotypic feature of a host by simple means are provided.

According to an embodiment of the invention said microbiome-specific data and said host-specific data can comprise microbiome-specific data modalities and host-specific data modalities. A large variety of data can provide the basis for an extra efficient and accurate prediction of the phenotypic feature to be predicted.

Within a further embodiment the provided or collected microbiome-specific and host-specific data or data modalities can be provided or collected from one or more databases. This provides a very reliable and controllable use of data or data modalities, wherein the content of the one or more databases can be updated periodically or as a response to an individual request by a user. In a further embodiment for each set of data or data modalities a certain target phenotypic feature to be predicted and/or a ground truth phenotypic feature can be also stored in the database. This can result in an extra efficient and accurate prediction of a phenotypic feature of a host by simple means.

According to a further embodiment the method can support regression and/or classification tasks. Thus, a very comfortable method and system are provided. In a further concrete embodiment the result of predicting the phenotypic feature can be a regression or a classification depending on individual situations or requests by a user.

Within a further embodiment the method can comprise encoding all provided microbiome-specific and host-specific data or data modalities with at least one—for example stochastic—encoder. This can provide a reliable basis for joining said microbiome-specific and host-specific data or data modalities in a suitable way. This encoding process can comprise encoding all provided microbiome-specific and host-specific data or data modalities to a definable space for enabling a reliable and comfortable handling of the encoding result. In a further embodiment the at least one encoder can comprise a derivable parametric function capable of learning time series. Such an encoder can play an important role in providing an extra efficient and accurate prediction of a phenotypic feature of a host by simple means.

Within a further embodiment the result of the encoding, particularly the joint representation, can be modeled for encompassing at least one interaction between single data or single data modalities. Such a modeling process can provide a very comfortable and flexible method and system considering individual application situations.

According to a further embodiment at least one machine learning model or at least one machine learning algorithm can learn to predict the phenotypic feature via multimodal learning and/or is designed for a multimodal variational approximation. On the basis of such a machine learning model or machine learning algorithm a particular efficient and accurate prediction of the phenotypic feature of the host to be predicted is possible. In a further embodiment this can be performed via a product of single-modality posteriors and in a further embodiment as a Multimodal Variational Information Bottleneck. The result of such a learning and prediction is an extra efficient and accurate prediction of a phenotypic feature of a host by simple means.

Within a further embodiment a training phase of the at least one machine learning process or machine learning algorithm with the microbiome-specific and host-specific data or data modalities can comprise collecting a target phenotypic ground truth associated with said microbiome-specific and host-specific data or data modalities. This will result in a very reliable and accurate prediction of the phenotypic feature to be predicted.

According to a further embodiment the training phase can further comprise computing single-modality posteriors via preferably stochastic encoding functions. This provides the basis for a very reliable and efficient joining of said microbiome-specific and host-specific data or data modalities and thus to an accurate prediction of the phenotypic feature.

Within a further embodiment the single-modality posteriors of the various data or data modalities can be joined in a multimodal joint posterior distribution. This can provide a high flexibility depending on individual application situations. In a further embodiment this joint posterior distribution or joint representation can be used to operate the phenotypic prediction by means of a decoder. An extra efficient and accurate prediction of a phenotypic feature of a host is the result of such a proceeding.

According to a further embodiment the training phase can further comprise training the machine learning model or machine learning algorithm by minimizing an objective function which accounts for an error score between prediction and ground truth. This feature provides a very efficient training and thus a very efficient and accurate prediction of the phenotypic feature to be predicted.

Within a further embodiment the method can comprise an inference phase comprising collecting microbiome-specific and host-specific input data or input data modalities for a new host, computing single-modality posteriors via stochastic encoding functions on the basis of the input data or input data modalities, joining the single-modality posteriors of the various data or data modalities in a multimodal joint posterior distribution or joint representation and using the multimodal joint posterior distribution or joint representation to operate the phenotypic prediction with the decoder. This combination of method steps provides the basis for a very reliable and extra efficient and accurate prediction of a phenotypic feature of a host by simple means.

In a further embodiment the method can be model/algorithm-agnostic and/or phenotype-agnostic. Such a method can be applied with high flexibility in different application and use situations.

According to a further embodiment the data or data modalities or input data or input data modalities can comprise at least one species-level abundance profile or species-level relative abundance profile, at least one strain-level marker profile and/or at least one host data modality. This will provide a reliable basis for performing the method and for providing an extra efficient and accurate prediction of a phenotypic feature of a host by simple means.

Advantages and aspects of embodiments of the present invention are described as follows:
1. Embodiments can (A) use microbiome-specific and host-specific data modalities to predict a phenotypic feature of the host, by (B) first encoding all input modalities to a latent space with stochastic encoders, and then (C) modeling the latent joint stochastic representation thus (D) encompassing the interactions between single modalities.
2. Further embodiments can use Multimodal Variational Information Bottleneck, a novel algorithm which allows for a multimodal variational approximation of [7] via a product of single-modality posteriors, see for example equation 5 in this document.

According to a further embodiment a method for predicting host phenotypic features based on multimodal microbiome-specific and host-specific features can comprise the steps of:

### Training Phase

1) Collecting microbiome-specific and host-specific training input data (1.A).
2) Collecting a target phenotypic ground truth associated with the target input data (1.A).
3) Computing single modality-posteriors via stochastic encoding functions (1.B).
4) Joining the single-modality posteriors of the various data modalities in a multimodal joint posterior distribution (1.C-1.D).
5) Using the joint representation to operate the phenotypic prediction by means of a decoder (1.A).
6) Training the model by minimizing an objective function which accounts for an error score between prediction and ground truth (1.A-fitting the model).

### Inference Phase

1) Collecting microbiome-specific and host-specific input data for a new host.
2) Computing single modality-posteriors via stochastic encoding functions.
3) Joining the single-modality posteriors of the various data modalities in a multimodal joint posterior distribution.
4) Using the joint representation to operate the phenotypic prediction with the decoder.
   Further embodiments of the system according to the invention provide multimodal microbiome-based phenotype prediction systems allowing to combine, integrate and exploit multiple heterogeneous information modalities coming from both the host and their microbiome. To the best of our knowledge, no existing predictive pipeline operates this fusion to predict a phenotypic feature.

See DeepMicro [5], MetaPheno [9], microbiome-based cardiovascular disease prediction [10], PopPhy-CNN [11]. These state-of-the-art disease prediction pipelines do not include any host-specific information to operate the prediction.

See DeepMicro [5], MetaPheno [9], microbiome-based cardiovascular disease prediction [10], PopPhy-CNN [11]. None of the considered pipelines support multimodal learning. Not only is host information ignored, but also one single microbiome data modality at a time can be input.

See DeepMicro [5]. This state-of-the-art approach presents a cumbersome 2-steps learning approach in which first embeddings are created, and then a downstream operates a classification. Our system and method support end-to-end machine learning algorithmic implementations.

Embodiments of the present invention provide a method and a system to operate microbiome-based phenotype prediction. Such a system efficiently joints multiple microbiome-specific and host-specific data modalities by computing a highly descriptive joint representation. This can be utilized for the prediction of a certain phenotypic feature of the host.

Further embodiments of the present invention can provide a system and method for multimodal microbiome-based phenotype prediction.

In light of the most recent discoveries on the microbiome's role on human health and diseases—briefly summarized at the beginning of this document—embodiments of the present invention provide prediction methods and systems aimed at inferring human phenotypic feature—e.g. the probability to develop a pathology, metabolism and optimal dietary characteristics, the effect of a therapy, the presence of a disease etc.—considering host data and also microbiome data. Such embodiments can provide an optimal fusion of multiple heterogeneous host-specific and microbiome-specific data modalities in order to operate a holistic phenotypic prediction. This allows the methods and systems to combine not only heterogeneous microbiome data modalities, e.g. species-relative and strain markers profiles, but also host-specific data modalities, e.g. electronic health records, EHR, radiological images, free-text written by physicians, life-style data.

Embodiments of this invention provide methods and systems which can predict one or more phenotypic features based on microbiome-specific and host-specific heterogeneous data or data modalities. Embodiments of the methods and systems can be agnostic with respect to the target phenotypic feature, i.e. they allow for maximum flexibility and do not place any constraint on what the users wish to predict. Embodiments of the invention can rely upon machine learning algorithms and can support both regression and classification tasks.

There are several ways how to design and further develop the teaching of the present invention in an advantageous way. To this end it is to be referred to the following explanation of examples of embodiments of the invention, illustrated by the drawing.

Where embodiments of the present invention address the set of microorganisms living in a host's large intestine—gut microbiota—together with information on their genome, reference is made to the microbiome. Since embodiments of this invention can comprise methods and systems which combine both data modalities, reference is made to the microbiome in the rest of this manuscript.

This section describes embodiments of the invention. These embodiments comprise for example a method and system for multimodal microbiome-based phenotypic prediction. The description of these embodiments of the invention is carried out in a phenotype-agnostic fashion, as the method does not place constraint on what phenotypic features are actually learned. Furthermore, since the method and system can implement various algorithms to operate, this section does not place emphasis on a specific machine learning approach. In the following embodiment descriptions, without lack of generality, multiple concrete implementations are presented, together with various algorithmic approaches and use cases.

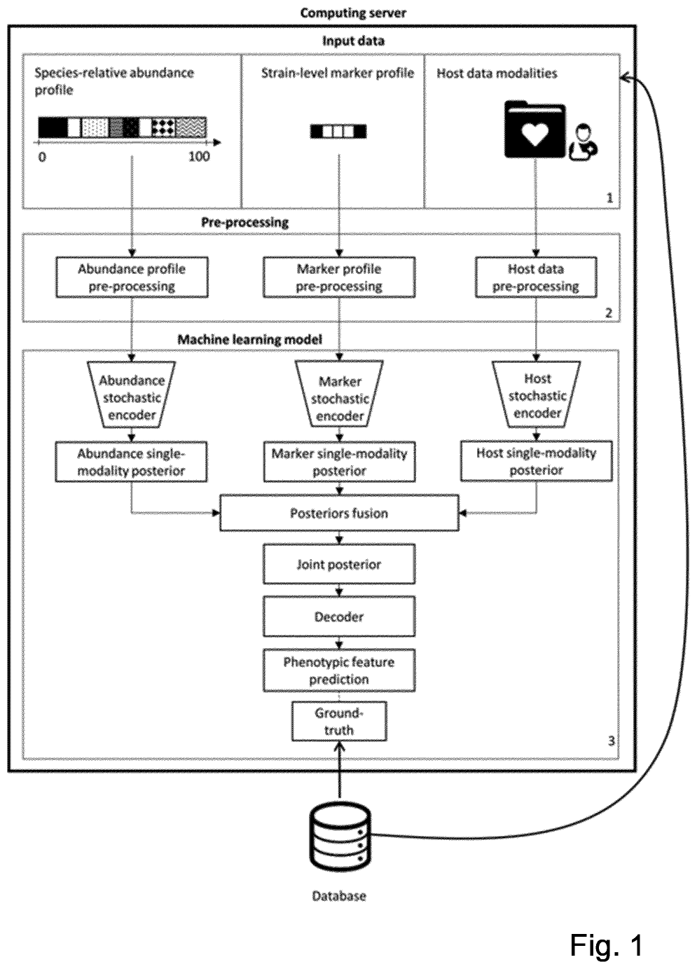

An important novel feature proposed by this invention lies in the combination of microbiome-specific and host-specific data to enable a holistic prediction of a phenotypic feature of the host. This allows to take into account complex and unknown interaction dynamics between the host, for example a human body, and the microbial community living in it. FIG. 1 depicts a system and in the following each part of the system is described.

In a dedicated computing server of the system, a machine learning model is trained to combine multiple heterogeneous host-specific and microbiome-specific data modalities. The data modalities are made available to the computing server from a database. For each patient/subject/host, the database stores the various data modalities, together with the associated ground truth phenotypic features. The machine learning model learns to predict the phenotype via multimodal learning.

### Input Data

For every patient/subject/host, multiple heterogeneous data modalities are stored in a database, some of which are microbiome-specific, while others are host-specific. FIG. 1 presents two different microbiome-specific data modalities: a species-level relative abundance profile and a strain-level marker profile. The former presents a continuous value for each identified microbial species which defines the evenness of distribution of individuals among species in a microbial community. The latter presents a binary value for each identified microbial gene marker. These are two commonly used descriptors for the microbiome, but the system can be extended to an arbitrary number of modalities.

For each set of input data modalities, a certain target phenotypic feature—which will be predicted—is also stored in the database.

### Pre-Processing

Each data modality undergoes a dedicated pre-processing step, aimed at facilitating the machine learning algorithm. The concrete pre-processing approach generally depends on the nature of the data and on the use case. Normally adopted techniques are scaling, standardization and feature selection.

The following describes the Machine learning model of FIG. 1:
Notation: in the following, X, Y, Z are random variables; x, y, z are multidimensional instances of random variables.

### Stochastic Encoders

For each data modality, a dedicated parametric stochastic encoder parametrizes a probability distribution of the data in a latent space. The stochastic encoder for data modality i is represented as p(Z|xⁱ), where Z is the encoding and xⁱ is the input data. The concrete stochastic encoder can model any probability distribution the user might desire. Furthermore, the function that maps the input xⁱ to its latent probability distribution can be any tractable parametric function. In sake of clarity, assuming a Gaussian N(μⁱ, Σⁱ) with mean μⁱ and covariance Σⁱ as latent distribution, and using a neural network f( ) to model the distribution, N(f^{μ}(xⁱ), f^{Σ}(xⁱ)) is obtained where f^{μ}(xⁱ) are the outputs of the network which model μⁱ and f^{Σ}(xⁱ) are the outputs of the network which model Σⁱ.

### Posteriors Fusion

After computing the single-modality encodings for p(Z|xⁱ) all input modalities, the obtained distributions are used to compute a joint posterior of the encoding conditioned on all input data modalities. This accounts for all M input data modalities. The joint posterior is p(Z|x¹, . . . , x^{M}).

Assuming conditional independence for the M input data modalities, the posterior fusion implements:

$$p ( Z \vert {x}^{1} , … , {x}^{M} ) = \frac{p ( Z ) p ( {x}^{1} , … , {x}^{M} \vert Z )}{p ( {x}^{1} , … , {x}^{M} )} = \frac{p ( Z )}{p ( {x}^{1} , … , {x}^{M} )} {\prod }_{i = 1}^{M} p ( {x}^{i} \vert Z ) = \frac{{\prod }_{i = 1}^{M} p ( Z \vert {x}^{i} )}{{\prod }_{i = 1}^{M - 1} p ( Z )} \frac{{\prod }_{i = 1}^{M} p ( {x}^{i} )}{p ( {x}^{1} , … , {x}^{M} )} \text{} ∝ \frac{{\prod }_{i = 1}^{M} p ( Z \vert {x}^{i} )}{{\prod }_{i = 1}^{M - 1} p ( Z )}$$

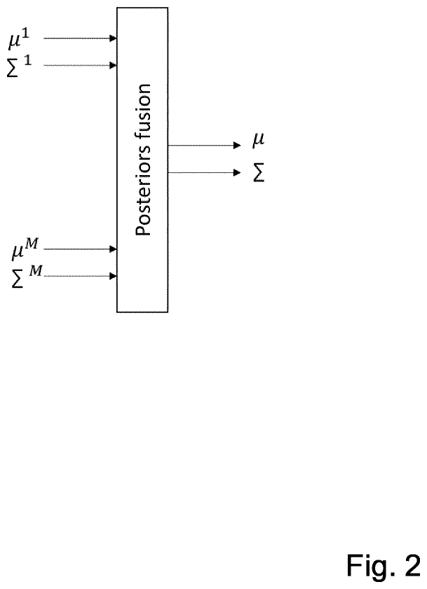

FIG. 2 depicts the fusion for the Gaussian case, i.e. each single-modality posterior is modeled as N(f^{μ}(xⁱ), f^{Σ}(xⁱ)). The fusion allows to compute (or approximate) the join posterior p(Z|x¹, . . . , x^{M})=N(μ,Σ).

FIG. 2 concretely shows in a diagram single-modality latent distributions p(Z|xⁱ) with i=1, . . . , M being fused to compute a joint posterior p(Z|x¹, . . . , x^{M})—Gaussian case.

### Decoder

The joint posterior is then used for the prediction. z is sampled from the joint-posterior distribution z˜p(Z|x¹, . . . , x^{M}). A parametric decoder models p(Y|z) and is used for operating the prediction, where Y is the target prediction.

### Target

The phenotype prediction can be either a regression or a classification. A supervised machine learning training paradigm is adopted to fit the model.

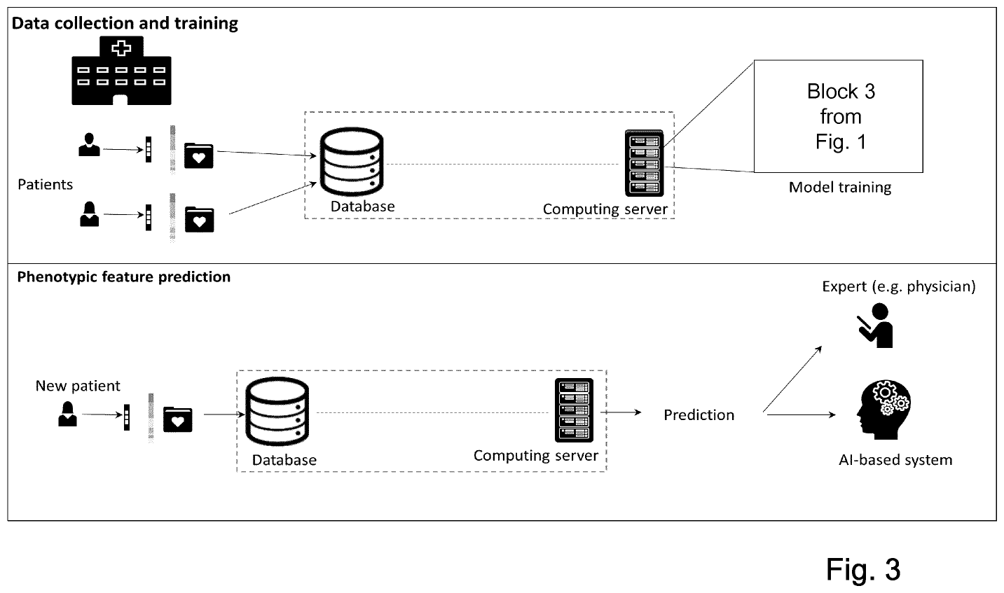

FIG. 3 depicts the real-world usage of the system, which will be contextualized in the two following embodiments. First, microbiome-specific and host-specific data needs to be collected and stored in a database. For each patient/subject/host, the ground truth phenotypic feature is also stored in the database. This could be, for instance, a diagnosis made by a physician. The collected data is then used to train the model. At inference time, the learned model is used to predict the phenotypic feature for a new patient. The prediction is then either analyzed by an expert (e.g. a physician) to support them in their work, or by an AI-based system which provides the patient with helpful suggestions e.g. on their lifestyle.

This can support e.g. the development of:
targeted dietary plans
precision-medicine therapies
diagnosis systems which account for the microbiome
optimal immunotherapies for personalized cancer vaccines

FIG. 3 depicts the main building blocks of the system—database and server for computation. Upper half: data collection and training of the model to learn predicting a certain phenotypic feature for a given set of patients. Lower half: after the training, inference of the learned phenotypic feature for a new patient.

### FURTHER EMBODIMENTS

### Embodiment A: Multimodal Microbiome-Based Disease Prediction

Recent studies show that microbiome data can be utilized to perform disease prediction [5]. This embodiment implements a solution for the same disease prediction task. By leveraging on the multimodal learning capabilities of the system, not only the microbiome is used, but both microbiome-specific and host-specific data are joint to infer whether a patient is affected by a disease.

As previously mentioned, the system is algorithm-agnostic and can implement any multimodal supervised learning algorithm. To better highlight how the system works, this section presents a concrete mathematical approach. The following is the description of a novel supervised machine learning algorithm aimed at learning a multimodal discriminative model via variational inference, named Multimodal Variational Information Bottleneck. The proposed algorithm is a novel generalization of [6] based on an information theoretic view of deep neural networks.

Remark: The embodiment does not necessarily have to implement this specific algorithm to solve the problem of the present invention.

Let Y be the target phenotypic feature associated with a set of input modalities X₁, . . . , X_{M}. Our goal is to learn a parametric probabilistic encoder p(Z|x₁, . . . , X_{M}; θ) that is maximally informative about our target Y, measured by the mutual information between our stochastic encoding Z and the target I(Z, Y; θ). In order to allow a more compact notation, let X be a random multidimensional random variable which comprises of all input modalities X₁, . . . , X_{M}, such that X=(X₁, . . . , X_{M}). The probabilistic encoder will therefore be expressed as p(z|x; θ).

The optimal parametric encoder can be obtained by maximizing the following objective, as defined by theory of the information bottleneck (IB) [7]:

$${R}_{IB} ( θ ) = I ( Z , Y ; θ ) - β I ( Z , X ; θ ) . \qquad ( 1 )$$

Intuitively, optimizing R_{IB}(θ) leads to learn an encoding Z that is maximally expressive about Y, while being maximally compressive about X. β≥0 controls the tradeoff. This forces Z to act like a minimal sufficient statistic of X for predicting Y. This approach is known as the information bottleneck (IB).

As derived in [6], assuming q(Y|Z) is a variational approximation of p(Y|Z), equation (1) can be rewritten as:

$${J}_{IB} = \frac{1}{N} {\sum }_{n = 1}^{N} {E}_{ϵ ∼ p ( ϵ )} [ - log q ( {y}_{n} \vert f ( {x}_{n} , ϵ ) ) ] + β KL [ p ( Z \vert {x}_{n} ) , r ( Z ) ] , \qquad ( 2 )$$

where f is a neural network which outputs both the mean of Z and its covariance matrix under the assumption that Z is Gaussian, KL is the Kullback-Leibler divergence, ϵ is a Gaussian random variable which allows for the reparametrization trick, r(Z) is a variational approximation of the true marginal p(Z). This formulation allows to directly backpropagate through a single sample of our stochastic parametric model and ensures that our gradient is an unbiased estimate of the true expected gradient.

The following is a novel generalization of the Deep Variational Information Bottleneck, which accounts for multiple data modalities. This general approach is named Multimodal Variational Information Bottleneck. On the right-hand side of equation (2), formulation of the neural network, which outputs both the mean of Z and its covariance matrix, is first generalized in order to explicitly account for all M data modalities:

$$f ( {x}_{n} , ϵ ) = f ( {x}_{n}^{1} , … , {x}_{n}^{M} , ϵ ) . \qquad ( 3 )$$

Furthermore, the formulation of the encoder can be rewritten to explicitly account for all data modalities:

$$p ( Z \vert {x}_{n} ) = p ( Z \vert {x}_{n}^{1} , … , {x}_{n}^{M} ) . \qquad ( 4 )$$

As shown by [8], the joint posterior p(Z|xₙ¹, . . . , xₙ^{M}) can be approximated by a product of single-modality posteriors, together with a prior p(Z):

$$p ( Z \vert {x}_{n}^{1} , … , {x}_{n}^{M} ) ∝ \frac{{\prod }_{i = 1}^{M} p ( Z \vert {x}_{n}^{i} )}{{\prod }_{i = 1}^{M - 1} p ( Z )} ∝ p ( Z ) {\prod }_{i = 1}^{M} \tilde{q} ( Z \vert {x}_{n}^{i} ) . \qquad ( 5 )$$

In Equation 5, the true single-modality posterior p(Z|xⁱ) is approximated by (Z|xⁱ)≡\tilde{q}(Z|xⁱ)p(Z).

This allows us to generalize the objective function (2) to account for the multimodal setting:

$${J}_{IB} = \frac{1}{N} {\sum }_{n = 1}^{N} {E}_{ϵ ∼ p ( ϵ )} [ - log q ( {y}_{n} \vert f ( {x}_{n}^{1} , … , {x}_{n}^{M} , ϵ ) ) ] + β KL [ p ( Z ) {\prod }_{i = 1}^{M} \tilde{q} ( Z \vert {x}_{n}^{i} ) , r ( Z ) ] . \qquad ( 6 )$$

Assuming the encoders \tilde{q} and the discriminator q(Y|Z) are neural networks, (6) can be optimized via stochastic gradient descent.

### Embodiment B: Multimodal Microbiome-Based Health Trajectory Prediction

The amount and variety of microorganisms exhibit high variance associated with our lifestyle. Embodiment B allows to extract patterns from the joint temporal dynamics of the microbiome-specific and host-specific data modalities. The problem solved by this embodiment consists in operating a self-regression of the patient EHR at a future time stamp.

### Input Data Streams

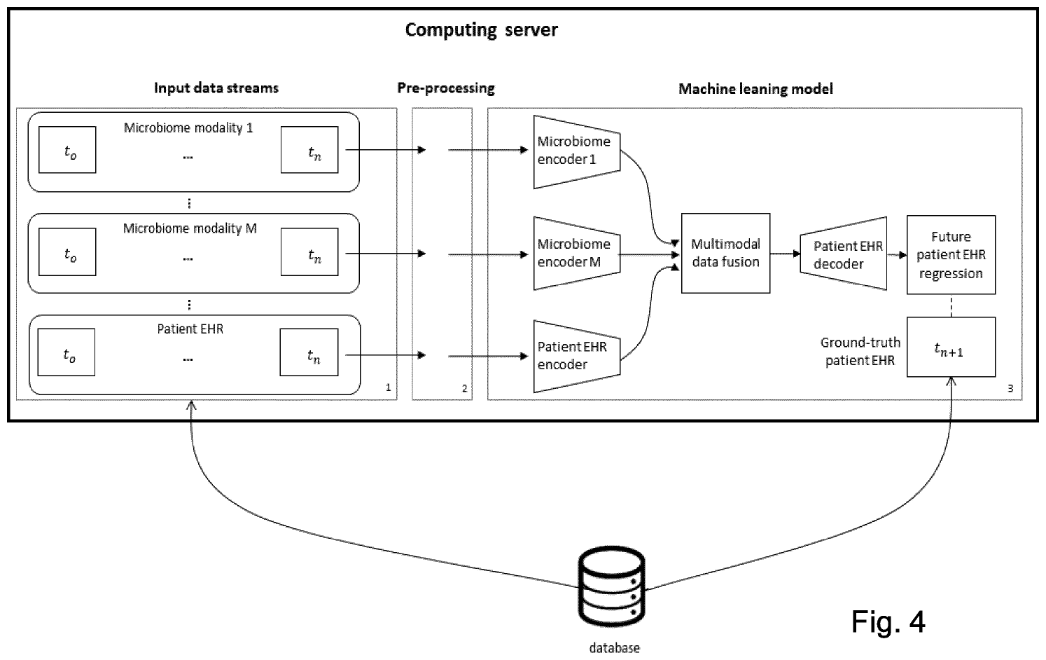

As depicted in FIG. 4, the input of the Embodiment B consists of data streams. The microbiome-specific data presents M different modalities. The host-specific data consists in a longitudinal EHR, captured at multiple time stamps.

### Pre-Processing

Data is pre-processed before being fed into the machine learning model.

### Machine Learning Model

For each modality, an encoding function is depicted in FIG. 4. These encoders can be any family of derivable parametric functions capable of learning time series, e.g. recurrent neural networks, RNN, long short-term memory cells, LSTM, gated recurrent units, GRU, Transformers. The system is agnostic with respect to which algorithm is actually implemented. FIG. 4 depicts the system in its “Health Trajectory Prediction” implementation.

After the encoding step, the data is fused, e.g. all hidden representations are concatenated, averaged or summed. A decoder operate a regression of the patient's EHR at a future time stamp tₙ₊₁, as a function of all data modalities observed in a preceding time window [t₀, tₙ].

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A method for predicting a phenotypic feature of a host based on a microbiome of the host by a data processing system, the method comprising:
providing or collecting microbiome-specific data and host-specific data;
joining the microbiome-specific data and the host-specific data by computing a joint representation; and
predicting the phenotypic feature on the basis of the joint representation using at least one machine learning model or machine learning algorithm.

2: The method according to claim 1, wherein the microbiome-specific data and the host-specific data comprise microbiome-specific data modalities and host-specific data modalities.

3: The method according to claim 2, wherein the provided or collected microbiome-specific and host-specific data or data modalities are provided or collected from one or more databases, wherein for each set of data or data modalities a certain target phenotypic feature to be predicted and/or a ground truth phenotypic feature is also stored in the database.

4: The method according to claim 1, wherein the method supports regression and/or classification tasks, wherein the result of predicting the phenotypic feature is a regression or a classification.

5: The method according to claim 2, wherein the method comprises encoding all provided microbiome-specific and host-specific data or data modalities with at least one stochastic encoder to a definable space, wherein the at least one stochastic encoder comprises a derivable parametric function capable of learning time series.

6: The method according to claim 5, wherein the joint representation results from the encoding and is modeled for encompassing at least one interaction between single data or single data modalities.

7: The method according to claim 1, wherein at least one machine learning model or at least one machine learning algorithm learns to predict the phenotypic feature via multimodal learning and/or is designed for a multimodal variational approximation via a product of single-modality posteriors.

8: The method according to claim 2, wherein a training phase of the at least one machine learning process or machine learning algorithm with the microbiome-specific and host-specific data or data modalities comprises collecting a target phenotypic ground truth associated with the microbiome-specific and host-specific data or data modalities.

9: The method according to claim 8, wherein the training phase further comprises computing single-modality posteriors via stochastic encoding functions.

10: The method according to claim 9, wherein the single-modality posteriors of the various data or data modalities are joined in a multimodal joint posterior distribution, wherein the joint posterior distribution or joint representation is used to operate the phenotypic prediction using a decoder.

11: The method according to claim 8, wherein the training phase further comprises training the machine learning model or machine learning algorithm by minimizing an objective function which accounts for an error score between prediction and ground truth.

12: The method according to claim 1, wherein the method comprises an inference phase comprising collecting microbiome-specific and host-specific input data or input data modalities for a new host, computing single-modality posteriors via stochastic encoding functions on the basis of the input data or input data modalities, joining the single-modality posteriors of the various data or data modalities in a multimodal joint posterior distribution or joint representation and using the multimodal joint posterior distribution or joint representation to operate the phenotypic prediction with the decoder.

13: The method according to any claim 1, wherein the method is model/algorithm-agnostic and/or phenotype-agnostic.

14: The method according to claim 2, wherein the data or data modalities or input data or input data modalities comprise at least one species-level relative abundance profile, at least one strain-level marker profile and/or at least one host data modality.

15: A data processing system for carrying out a method for predicting a phenotypic feature of a host based on a microbiome of the host, the system:
providing or collecting means for providing or collecting microbiome-specific data and host-specific data;
joining means for joining the microbiome-specific data and the host-specific data by computing a joint representation; and
predicting means for predicting the phenotypic feature on the basis of the joint representation by means of at least one machine learning model or machine learning algorithm.

16: The method according to claim 1, wherein the method provides support for development of optimized immunotherapies for personalized cancer vaccines.
